(19) United States
(12) Patent Application Publication
(10) Pub. No.: US 2007/0276113 A1
(43) Pub. Date: Nov. 29, 2007

(54) Grafted Polymer Comprising a Polyorganosiloxane Backbone and Gylcoside Units

(76) Inventor: Etienne Fleury, Irigny (FR)

Correspondence Address: Jean-Louis Seugnet;Rhodia Inc., 8 Cedar Brook Drive, CN 7500, Cranbury, NJ, 08512-7500, US

(21) Appl. No.: 10/592,159

(22) PCT Filed: Mar. 10, 2005
(86) PCT No.: PCT/FR05/00577

§ 371 Date: Jun. 25, 2007

(30) Foreign Application Priority Data
Mar. 10, 2004 (FR) 0402497

(51) Int. Cl. C08G 77/26; C07H 15/10; C08G 77/388

(52) U.S. Cl. 528/31; 528/38

(57) ABSTRACT

The invention relates to a grafted polymer comprising a polyorganosiloxane backbone and gylcoside units. The invention also relates to a method of preparing the polymer and to the use thereof. The invention further relates to an intermediate compound which is derived from a glycoside and which can be used to graft a polyorganosiloxane.

The present invention relates to a grafted polymer comprising a polyorganosiloxane backbone and glycoside units. The invention also relates to a method for preparing the polymer, and to the use thereof. The invention also relates to an intermediate compound derived from a glycoside, which can be used to graft a polyorganosiloxane.

Glycosides are compounds which are of specific interest due to their physicochemical properties. They are, in addition, compounds of natural origin which have a certain chemical complexity and which can be readily accessible. This origin may make them particularly attractive from an environmental and/or toxicological and/or commercial point of view. Numerous products which derive from glycosides have therefore been studied. It is thus known practice to functionalize glycosides in order to modulate the properties thereof.

It has, for example, been proposed to functionalize glycosides in order to subsequently be able to graft them onto polyorganosiloxane, in order to obtain polyorganosiloxanes with modified properties.

Functionalizations of an alcohol on the anomeric carbon of a glycoside, in the presence of an acid, under homogeneous conditions, are thus known. This reaction is known as a “Fischer reaction”. These functionalizations do not make it possible to obtain highly pure products. There is in particular a parasitic reaction, the Maïer reaction, which creates unsaturations in the product obtained. Such parasitic unsaturations can pose problems when the functionalized glycoside is used as a reactant in other reactions, in particular during grafting on a polymer. The parasitic unsaturations can, for example, cause unwanted crosslinkings. These parasitic reactions can require purification steps which can prove to be expensive.

Methods for grafting polyorganosiloxane with glycosides functionalized on the anomeric carbon using compounds comprising an alcohol group and an allyl group are in particular known. Such methods are, for example, described in the following documents:
Carbohydrate modified polysiloxanes II, G. Jonas et al., Acta Polymer, 45, 14-20 (1994),
Preparation of oligomethylsiloxanes with sugar moiety at a terminal group as transdermal penetration enhancer, Akimoto et al., Macromol. Chem. Phys. 2000, 201, 2729-2734,
Silicon-modified Carbohydrate Surfactants I, R. Wagner, Applied organometallic chemistry, vol. 10, 421-435 (1996).
published patent application EP 612759.

Such methods lead to compounds comprising organosiloxane units and grafted glycoside units, the organosiloxane units and the glycoside units being connected via a linking unit comprising an —O— group. These methods are, however, too complex to seriously envision an industrial exploitation thereof, and/or too expensive to seriously envision uses in which properties that they could provide would compensate for the cost thereof.

A method for functionalizing glycosides with allylamine is, moreover, known. Such a method is, for example, described in the following documents:
An efficient synthesis of N-allylglycosylamides from unprotected carbohydrates, J. Org. Chem. 1996, 61, 3417-3422.
Chemoenzymatic synthesis of neoglycopeptides: Application to an alpha-Gal-terminated neoglycopeptide, J. Org. Chem. 2001, 66, 2948-2956.

An object of the present invention is to provide a novel method for preparing grafted polymers comprising a polyorganosiloxane backbone and glycoside units. This method is in particular sufficiently simple to envision an industrial exploitation thereof, and/or to seriously envision uses in which properties that the grafted polymers could provide would compensate for the cost thereof. Thus, a subject of the invention is also novel polymers which can be obtained by means of the method. Finally, a subject of the invention is also an intermediate product which can be used to carry out the method.

The invention thus provides a grafted polymer having one of the following formulae:

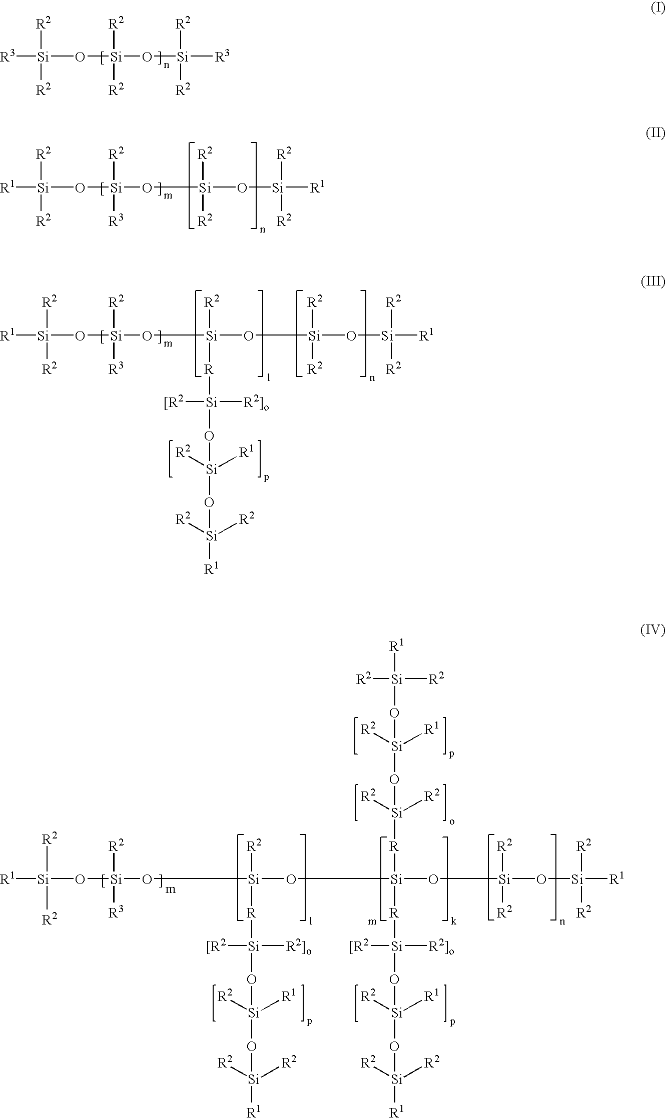

in which:
R², which may be identical or different, is a hydrocarbon group, preferably a methyl group,
R³, which may be identical or different, is a group of formula -L-G in which:
   L is a divalent linker group comprising a nitrogen atom,
   G is a glycoside comprising —OH groups,
R¹, which may be identical or different, is an R² or R³ group,
R is a divalent group comprising an oxygen atom, preferably an —O— group,
n is an average number other than 0.
m is an average number greater than or equal to 0,
k and l are average numbers greater than or equal to 0, and
o and p, which may be identical or different, are average numbers greater than or equal to 0.

The polymer can in particular be:
a compound of type G-MDₙM-G of formula (I) where n is between 2 and 100, for example equal to 10, 13 or 29, and R² is a methyl group,
a compound of type M(D-G)M of formula (II) where m=1, n=0, R² is a methyl group and R¹ is a methyl group.

The invention also provides a method for preparing the grafted polymer, comprising the following steps:

a) preparing a graft compound of formula L′-G′, in which:
L′ is a group comprising a group that is ethylenically unsaturated, and comprising a nitrogen atom, and
G′ is a glycoside comprising optionally protected —OH groups,

b) grafting the graft compound by means of a method comprising a hydrosilylation step, and

c) optionally, deprotecting the —OH groups.

The invention also provides a compound (graft compound) which can be used in the preparation of the grafted polymer, and which has the following formula:
G″-N(COCH₃)—CH₂—CH═CH₂

in which G″ is a glycoside connected to the N atom via an anomeric carbon, the —OH groups of the glycosides being at least partially protected with acetyl groups —COCH₃.

Definitions

Glycoside

In the present application, the term “glycoside” refers to any group comprising one or more glycoside units, and to the derivatives of these groups. When the glycoside comprises several glycoside units, the term “polyglycoside” is also used. The term “polyglycoside” is intended to mean a glycoside comprising at least two glycoside units.

The glycoside units, the glycosides, the polyglycosides, the derivatives thereof, and the structures and formulae thereof are known to those skilled in the art. With respect to the glycoside units, it is specified that they may in particular be aldoses, ketoses, or derivatives, with rings containing 5 atoms (pentoses) or 6 atoms (hexoses). It is also known to those skilled in the art that the glycosides, the polyglycosides and the derivatives thereof have an “anomeric carbon” at one end, the right according to written convention. It is also known that the glycoside units, the glycosides, the polyglycosides and the derivatives thereof have —OH groups (if the latter are not protected).

The glycosides include in particular:
methylglycosides, glycosides comprising a —COOH group,
monoglycosides and polyglycosides.

By way of examples of monosaccharide glycosides, mention may be made of the following glycosides:

D-glucose, fructose, sorbose, mannose, galactose, talose, allose, gulose, idose, glucosamine, mannoamine, galactosamine, glucuronic acid, rhamnose, arabinose, galacturonic acid, fucose, xylose, lyxose, ribose and palatinose.

By way of examples of disaccharide or oligosaccharide glycosides, mention may be made of the following glycosides:
disaccharides: maltose, gentiobiose, lactose, cellobiose, isomaltose, melibiose, laminaribiose, chitobiose, xylobiose, mannobiose, sophorose,
oligosaccharides: maltotriose, isomaltotriose, maltotetraose, maltopentaose, xyloglucan, maltoheptaose, mannotriose, manninotriose, maltodextrins, chitotriose, in general disaccharides or oligosaccharides exhibiting β-1,4 linkages, starches having at least 5 dextrose equivalents.

By way of examples of glycosides, mention may also be made of:
starch derivatives, in particular maltose, maltodextrins,
galactomannans and derivatives thereof, for example guar polymers and derivatives thereof, obtained by hydrolysis of natural guar, and optionally chemical modification (derivatization). Natural guar is extracted from the albumen of certain plant seeds, for example Cyamopsis tetragonalobus. The guar macromolecule consists of a linear main chain constructed from β-D-mannose monomeric sugars connected to one another via (1-4) linkages, and α-D-galactose side units connected to the β-D-mannoses via (1-6) linkages.

The polyglycosides, comprising several glycoside units, can be described as chains of glycosides (mono- and/or polyglycosides). In the present application, a glycoside chain is described by the formula G^{a}-G^{b}-, in which G^{a} is a glycoside or a polyglycoside, and G^{b} is a glycoside or a polyglycoside. When G^{a} or G^{b} is a polyglycoside, the latter can also be described by a formula G^{a′}-G^{b′}-, in which G^{a′} is a glycoside or a polyglycoside, and G^{b′} is a glycoside or a polyglycoside, and so on. Glycosides or polyglycosides that can constitute G^{a}, G^{b}, G^{a′}, G^{b′}, etc., groups have been mentioned above.

Protected Glycoside

The term “protected glycoside” is intended to mean a glycoside in which at least some of the —OH groups have been modified to groups of formula —OP, where P is a protective group, for example a trimethylsilyl (TMS, —Si(CH₃)₃), or an acetyl group —COCH₃.

The protective group can have the function of preventing unwanted side reactions on the —OH groups of the glycoside, for example reactions leading to crosslinkings during the reaction with the backbone compound.

The protective group can also help to modulate the solubility of the glycoside.

The protection is said to be complete if all the —OH groups of a glycoside are substituted with the —OP group.

The protection is said to be partial if only some of the —OH groups of a glycoside are substituted with the —OP group.

Details of the method according to the invention are given below.

Preparation of the Graft Compound

In a first step a) of the method, a graft compound of formula L′-G′ is prepared, in which formula:
L′ is a group comprising a group that is ethylenically unsaturated, and comprising a nitrogen atom, and
G′ is a glycoside comprising —OH groups which are optionally protected, with a glycoside G mentioned later in this application.

The group L′ preferably has one of the following formulae:
—NR⁴—(CH₂)_{q′}—CH═CH₂
—NR⁴—(CH₂)_{q′}—C≡CH

in which:
R⁴ is a hydrogen atom, an alkyl group or a protective group, for example an acetyl protective group —COCH₃, and
q′ is an integer greater than or equal to 0.

The L′ group is thus preferably an allylamine-derived group, with preferably an acetyl protective group connected to the nitrogen atom.

Thus, the graft compound can be a compound of formula G′-N(COCH₃)—CH₂—CH═CH₂, in which G′ is an optionally protected glycoside. The protective groups can be chosen from trimethylsilyl (-TMS, —Si(CH₃)₃) or acetyl (—COCH₃) groups.

According to an advantageous embodiment, G′ has the formula G^{a}-G^{b}- in which G^{a} is a glycoside (monoglycoside or polyglycoside) and G^{b} is linker glycoside connected to the N atom via an anomeric carbon. G′ may, for example, be a cellobiose group, or a G^{a}-G^{b}-group in which G^{b} is a cellobiose group.

The graft compound can be prepared by reaction of the anomeric carbon of a glycoside G″ with a functionalization reactant comprising an amine group, preferably a primary amine group, and a group comprising an unsaturation (for example, alkene or alkyne type). The glycoside G″ comprises free —OH groups. The functionalization reactant is preferably allylamine.

The reaction can be carried out in the absence of solvent, at ambient temperature, but other reaction methods are not excluded.

Thus, step a) for preparing a graft compound can comprise the following steps:

a1) reaction of the anomeric carbon of a glycoside G″ comprising free —OH groups with excess allylamine,

a2) elimination of the excess allylamine,

a3) reaction with acetic anhydride, so as to protect the nitrogen atom, and optionally primary —OH groups of the glycoside, and

a4) optionally, protection of the other —OH groups of the glycoside.

An example of preparation of a graft compound in which the glycoside G″ is cellobiose, by reaction with an allylamine functionalization compound, is given below. The example below comprises steps a1) to a3), in which —P represents an acetyl group:

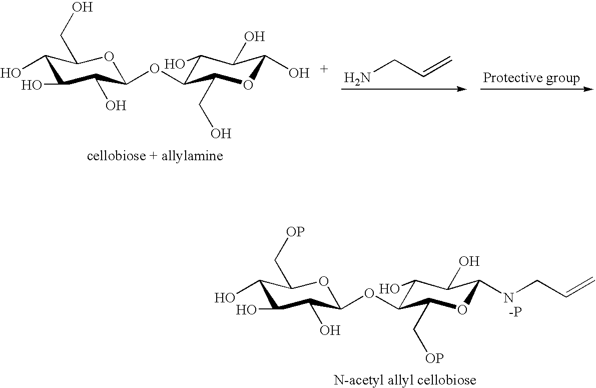

According to a particularly advantageous embodiment of the invention, step a3) consisting of reaction with acetic anhydride is carried out under conditions such that at least some of the —OH groups of the glycoside are acetylated, in addition to the nitrogen atom. The acetylation is, for example, carried out at ambient temperature in the presence of acetic anhydride for several hours. The hydroxyl functions in the C₆-position are optionally modified if there is an excess of reactant. In this embodiment, the primary —OH groups are acetylated as a priority. It is not impossible for all the —OH groups to be acetylated. This embodiment can make it possible in particular to avoid using a specific protection step. It is advantageous with regard to the simplicity of the method, and with regard to the control of the solubility of the graft compound.

Thus, according to an advantageous embodiment, the graft compound is a group of formula:
G″-N(COCH₃)—CH₂—CH═CH₂

in which G″ is a glycoside connected to the N atom via an anomeric carbon, the —OH groups of the glycosides being at least partially protected with acetyl groups —COCH₃. According to an advantageous embodiment, G″ is a cellobiose group, or a G^{a}-G^{b}-group in which G^{b} is a cellobiose group, and G^{a} is a glycoside.

Optional Protections

The preparation of the graft compound can include a protection. According to one embodiment, the protection can be carried out during a specific protection step a4). The specific protection step can be a partial or complete protection of the G′ group with acetyl groups, for example using acetic anhydride, in a solvent such as pyridine, or using acetic acid in the presence of strong acid of the H₂SO₄ type. The specific protection step can be a partial or complete protection of the G′ group with trimethylsilyl groups, for example using trimethylchlorosilane, in a medium of imidazole/toluene type, or preferably in pyridine medium.

An example of protection step a4) with acetyl groups is given below:

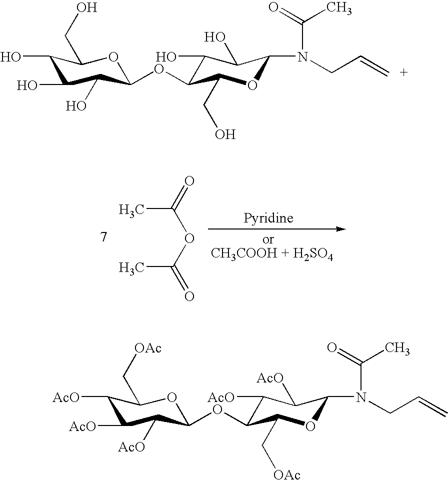

An example of protection step a4) with TMS groups is given below:

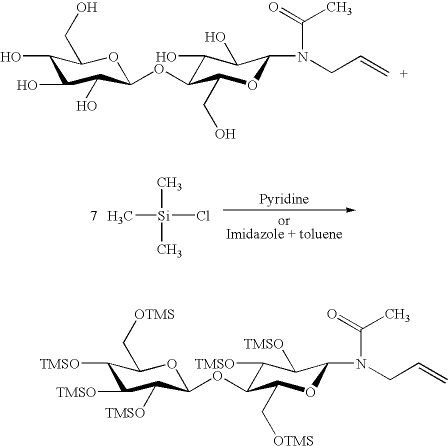

Grafting

The grafting is carried out during a step b) by means of a method comprising a hydrosilylation step.

According to a first embodiment, step b) is grafting onto a backbone compound, by means of a simple hydrosilylation reaction.

According to a second embodiment, step b) comprises a reaction consisting of grafting onto a backbone compound by means of hydrosilylation, and a redistribution reaction. The redistribution reactions are known to those skilled in the art.

According to a third embodiment, step b) comprises hydrosilylation of the graft compound with a backbone silane having the formula H—Si(OR⁵)₃₋ₐR⁵ₐ, in which R⁵, which may be identical or different, is a hydrocarbon group, and a is equal to 1 or 2, the backbone silane preferably having the formula H—Si(—O—CH₂—CH₃)₂CH₃, and then redistribution in the presence of a polyorganosiloxane. Such functionalized silane redistributions are known to those skilled in the art.

Thus, step b) can be described in the following way:

grafting the graft compound by means of a method comprising a hydrosilylation step, said method comprising:
hydrosilylation of the graft compound with a backbone compound having one of the following formulae:

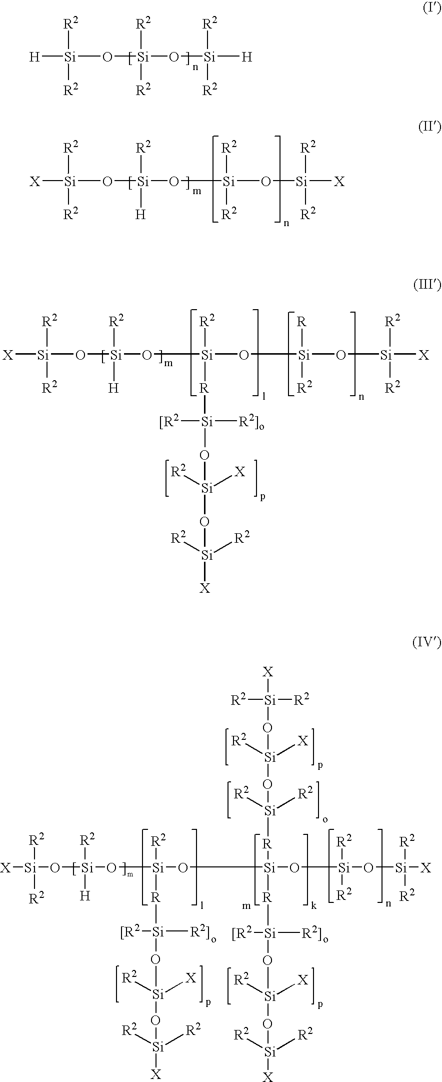

in which:
R², which may be identical or different, is a hydrocarbon group, preferably a methyl group,
X, which may be identical or different, is a hydrogen atom or an R²,
R is a divalent group comprising an oxygen atom, preferably an —O— group,
n is an average number other than 0,
m is an average number greater than or equal to 0,
k and l are average numbers greater than or equal to 0, and
o and p, which may be identical or different, are average numbers greater than or equal to 0, the reaction being carried out in a solvent which solubilizes or swells the graft and the backbone, then, optionally, a redistribution, or
hydrosilylation of the graft compound with a backbone silane having the formula H—Si(OR⁵)₃₋ₐR⁵ₐ, in which R⁵, which may be identical or different, is a hydrocarbon group, and a is equal to 1 or 2, the backbone silane preferably having the formula H—Si(—O—CH₂—CH₃)₂CH₃, the reaction being carried out in a solvent which solubilizes or swells the graft and the backbone, and then a redistribution in the presence of polyorganosiloxane.

The backbone compound can in particular be:
a compound of type M′DₙM′ of formula (I′) where n is between 2 and 100, for example equal to 10, 13 or 29, and R² is a methyl group,
a compound of type MD′M of formula (II′) where m=1, n=0 and R² is a methyl group.

Hydrosilylation Reaction

During this phase, the graft compound is reacted with a backbone compound.

The backbone compound is a polyhydrogenosiloxane or a hydrogenosilane. Such compounds comprise a variable number of hydrogen atoms, at the ends of the macromolecular chains, and/or within the macromolecular chains, or a hydrogen atom connected to the silicon atom of a silane.

Such hydrogenated compounds are known to those skilled in the art and are commercially available. In the present application, in addition to the formulae mentioned above, reference may also be made to the terminology known to those skilled in the art for M, D, T and Q groups and corresponding M′ and D′ groups, comprising a hydrogen atom connected to a silicon atom, or a graft.

The hydrosilylation reaction is also known to those skilled in the art. Many works exist on this subject. It consists in reacting a hydrogen atom connected to a silicon atom, with a group comprising an unsaturation. The hydrosilylation reaction is generally carried out using a catalyst, for example a platinum-based catalyst, such as the Karstedt catalyst described in patent U.S. Pat. No. 3,775,452.

The hydrosilylation process is preferably carried out:
at a temperature of 50 to 150° C., preferably of 50 to 100° C., most particularly of 60 to 90° C.,
in the presence of a solvent (S), which is inert with respect to the reactants, and which has, at atmospheric pressure, a boiling point below 250° C., preferably below 150° C.

The hydrosilylation process is carried out in the presence of a hydrosilylation catalyst; the latter is in particular chosen from those which are based on platinum (0) or on a derivative of platinum (0), such as the platinum complexes described in U.S. Pat. No. 3,159,601, U.S. Pat. No. 3,159,662, U.S. Pat. No. 3,715,334, U.S. Pat. No. 3,814,730, etc. A preferred catalyst is the KARSTEDT catalyst, used, for example, at a rate of 1 to 300 parts, preferably of 5 to 100 parts by mass of platinum per million parts by mass of reactants (SiH) and (Vi) used.

The hydrosilylation process is preferably carried out under atmospheric pressure.

The introduction of the reactants (backbone compound and graft compound) is preferably carried out by simultaneously running the two reactants continuously into the reaction mass comprising the solvent and the catalyst.

The solvent (S) and the reactants which have not reacted can subsequently be eliminated. The elimination thereof can be carried out by distillation under vacuum or reduced pressure (for example, of the order of 1.013 Pa to 101 300 Pa).

This distillation process can optionally be followed by a hydrogenation process.

According to one embodiment, the medium derived from the hydrosilylation process is subjected to a hydrogenation process according to the conditions described above (in order to reduce the amount or eliminate the presence of unsaturated compounds resulting from the hydrosilylation reaction), and is then optionally subjected to a process consisting in eliminating the products other than the desired compounds. This elimination process can be carried out by distillation under vacuum or reduced pressure, for example of the order of 1.013 Pa to 101 300 Pa.

Solvent

The hydrosilylation reaction is carried out in a solvent which solubilizes or swells the graft and the backbone.

The solvent may, for example, be chosen from the following solvents:
aprotic polar solvents, preferably DMF, DMAc,
protic polar solvents, preferably methanol, IPA (isopropyl alcohol), TbuOH, and
apolar solvents, preferably toluene, hexane, xylene.

According to an advantageous embodiment, the solvent is IPA and the groups are protected with acetyl groups —COCH₃ in step a).

Examples of reaction schemes for grafting, onto a backbone compound, a graft compound comprising cellobiose, protected with TMS, are given below.

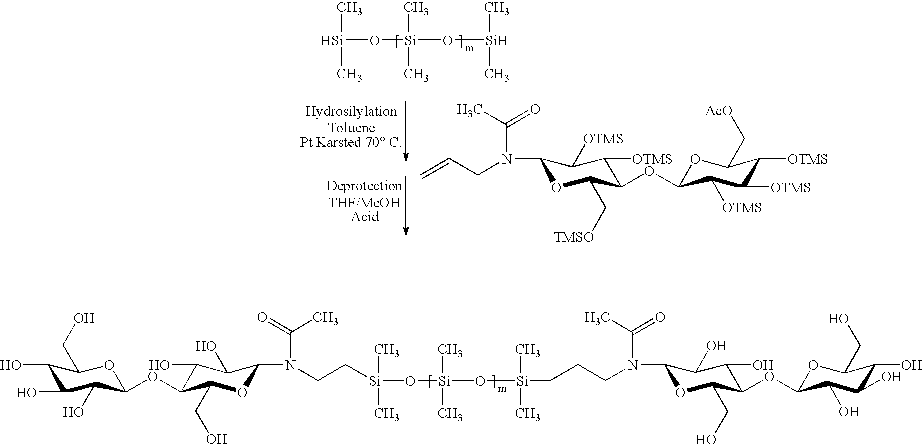

According to the first embodiment, mentioned above, step b) can be a simple reaction consisting in grafting the graft compound onto the backbone compound, by hydrosilylation.

According to the second embodiment, a hydrosilylation reaction of the type such as that mentioned above can be carried out, and then a redistribution can be carried out. This method consists in preparing, firstly, a polyorganosiloxane oligomer containing glycoside groups by hydrosilylation according to the route described above and in subsequently carrying out a redistribution. The redistribution thus makes it possible to adjust the length of the chains. It can be carried out by reaction of polyorganosiloxane containing glycoside groups with octamethylcyclotetrasiloxane (D4) in the presence of an acid catalyst, for example Tonsil (diatomaceous earth). The reaction is carried out in the presence or absence of solvent, preferably at a temperature of between 50 and 100° C. Advantageously, a backbone compound of M′DnM′ type with n between 0 and 20 is used as backbone compound, in order to obtain the polyorganosiloxane containing glycoside groups.

According to the third embodiment, a grafting onto an alkoxysilane or dialkoxysilane can be carried out by means of a hydrosilylation reaction, followed by a redistribution reaction in the presence of a polyorganosiloxane. In this approach, an organosilane can be used without modification or after prior hydrolysis. It is preferable to use a dialkoxysilane of the type G-L-Si(OR²)(R²) where R², which may be identical or different, is an alkyl group, preferably a dialkoxysilane of formula G-L-Si(OC₂H₅)(CH₃). In a first procedure, the silane can be introduced into a silicone oil and the redistribution is carried out at a temperature (70-110° C.) in the presence of a redistribution catalyst such as potassium silanolate. This catalyst is advantageously neutralized at the end of the reaction. Similarly, it is sometimes necessary to eliminate the cyclic compounds formed during the redistribution reaction. The chain ends are of the alkoxy or methyl type.

In a second procedure, the hydrolysis of the silane is carried out in the presence of water and the alcohol is eliminated. The redistribution is then carried out at temperature and in the presence of a basic catalyst.

Optional Deprotection

If the graft compound used for the reaction comprises —OP groups, i.e. protected —OH groups, the method can comprise a deprotection step. Such a step consists in carrying out a reaction which converts the —OP groups to —OH groups.

For example, trimethylsilyl groups (TMS, —Si(CH₃)₃) can be deprotected by reacting the grafted polymer at ambient temperature using a treatment consisting of methanolysis, possibly in the presence of an acidic resin, of the amberlite type.

Acetyl groups can be deprotected by reacting the grafted polymer using a treatment in methanol medium containing sodium methoxide, at ambient temperature, or else still in methanol medium in the presence of gaseous ammonia.

Details of the polymer according to the invention are given below.

The polymer according to the invention, which can be prepared by means of the method described above, has one of the formulae mentioned above. In these formulae:
m+n is advantageously between 0 and 1000, preferably between 0 and 100, the ratio of m to n being 1/1 and 1/100, preferably between 1/20 and 1/50, or
m+n+o+p is advantageously between 0 and 1000, preferably between 0 and 300, the ratio of n+o to m+p being between 1/1 and 1/100, preferably between 1/20 and 1/50.

The L group is preferably a divalent group having one of the following formulae:
—NR⁴—(CH₂)_{q}—CH₂—CH₂—
—NR⁴—(CH₂)_{q}—CH(CH₃)—
—NR⁴—(CH₂)_{q}—CH═CH—
—NR—(CH₂)_{q}—C(CH₃)═CH—

in which
R⁴ is a hydrogen atom, an alkyl group or a protective group, and
q is an integer greater than or equal to 0.

R³ is advantageously a group of formula G-NH—CH₂—CH₂—CH₂—, in which G has the formula G^{a}-G^{b}-, in which G^{a} is a glycoside (monoglycoside or polyglycoside), and G^{b} is a linker glycoside connected to the L group via an anomeric carbon. G can, for example, be cellobiose, or a group of formula G^{a}-G^{b}- in which G^{a}- is a glycoside (monoglycoside or polyglycoside), and G^{b} is cellobiose.

G is advantageously a glycoside chosen from:
glucose, fructose, sorbose, mannose, galactose, talose, allose, gulose, idose, glucosamine, mannoamine, galactosamine, glucuronic acid, rhamnose, arabinose, galacturonic acid, fucose, xylose, lyxose, ribose, palatinose,
maltose, gentiobiose, lactose, cellobiose, isomaltose, melibiose, laminaribiose, chitobiose, xylobiose, mannobiose, sophorose,
maltotriose, isomaltotriose, maltotetraose, maltopentaose, xyloglucan, maltoheptaose, mannotriose, manninotriose, maltodextrins, chitotriose,
starches having at least 5 dextrose equivalents, and
galactomannans and derivatives thereof.

Form or Presentation of the Grafted Polymer

The grafted polymer according to the invention can in particular be in the form of an oil. It can also be in a form dispersed or solubilized in a vector, for example at a concentration of 10 to 90% by weight. The vector can advantageously be a solvent for the polymer, for example an optionally volatile silicone compound, for example a linear or cyclic polydimethylorganosiloxane such as cyclopentasiloxane, disiloxane, linear dimethicones, or a trimethylsiloxyphenyl dimethicone, or a mixture.

Uses

The grafted polymer according to the invention can in particular be used as an emulsifier or coemulsifier for preparing or stabilizing emulsions. It can, for example, be used in emulsions in which one phase is a silicone oil. When in the form of a solution in polyorganosiloxane, for example in cyclopentasiloxane, it can be used as an emulsifier for water-in-oil or oil-in-silicone emulsions. It can also be used to render several compounds compatible within a formulation. It can also be used as an agent for aiding the deposition of another compound, or as an agent for initiating the deposition of another compound. It can be of use for delivering a silicone compound onto a surface. It can also be used as a dispersant or codispersant for preparing or stabilizing dispersions of particles, for example pigments.

It can in particular be used or included in a cosmetic formulation, possibly intended to be rinsed off, for skin care and/or hair care and/or lip care, for example in skincare creams, milks or oils, suncreams, milks or oils, shampoos, conditioners, shower gels, make-up compositions, lipsticks, deodorants. In particular, the polymer according to the invention has the advantages, in these applications, of being relatively non-irritant, of being partially biodegradable, of providing a pleasant feel, and/or of providing advantageous spreading.

Other details or advantages of the invention will emerge more clearly in view of the example given below by way of indication.

### EXAMPLE

Functionalization of Cellobiose:

3.26 g of cellobiose (9.51 mmol) and 15.22 g of allylamine (266.55 mmol) are introduced at ambient temperature into a 250 ml three-necked glass reactor equipped with a stirrer and a condenser. After dissolution of the cellobiose, the mixture is left stirring and under a layer of argon at ambient temperature for 72 hours.

The excess allylamine is then eliminated under vacuum by heating at 50° C. under vacuum. After elimination of most of the allylamine, 20 ml of toluene are introduced in order to carry out a codistillation under vacuum so as to completely eliminate the allylamine. After this treatment, 220 ml of a methanol/acetic anhydride mixture (10/1 v/v) are introduced into the reactor and this reaction medium is left under argon at ambient temperature for 16 hours. The reaction medium is then optionally filtered and then distilled under vacuum at 50° C. in order to eliminate the acetic anhydride. The product obtained, referred to as A, is an orangey solid, the ¹H NMR analysis of which confirms the expected structure and shows that the primary hydroxyl functions are partially acetylated.

The product is soluble in water, in DMSO or alternatively in isopropyl alcohol (IPA)

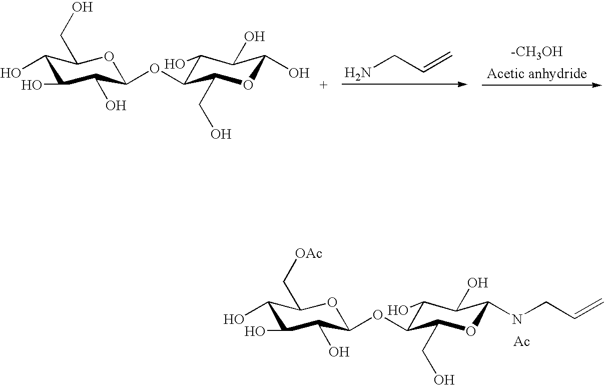

Silylation of Cellobiose Comprising an Allyl Group:

1 g of the product A prepared above, 20 ml of pyridine containing no water and then 2.33 g (21.5 mmol) of trimethylchlorosilane are introduced into a glass reactor. The reaction medium is maintained at ambient temperature for 16 hours. After this period of time, the pyridine is eliminated by distillation under vacuum after having added toluene. The product obtained, referred to as B, is analyzed by NMR. The presence of the Si—CH₃ peaks is clearly observed around 0 ppm and the signals of the allyl protons are clearly observed between 6 and 4 ppm.

Hydrosilylations:

1/Hydrosilylation on a per-O-silylated Product B:

1 g of per-O-silylated N-acetyl allylcellobiose (1.08 mmol) is dissolved in 20 ml of toluene in a three-necked reactor equipped with stirring and an argon inlet. 100 ppm of platinum (relative to the reactants) in the form of Karstedt platinum, are added and the medium is brought to 70° C. with stirring. 0.45 g of an α,ω-dihydrogen polyorganosiloxane oil of molar mass 1000-620H2 oil (i.e. 0.9 mmol of SiH) is then loaded into the reactor. The reaction is followed by measuring the SiH function content. After reaction for 4 hours, the degree of conversion is greater than 90%. 2.3 grams of carbon black are then added to the reaction medium in order to eliminate the catalyst. After filtration, the toluene is eliminated by distillation under vacuum. The product obtained (reference C) is in the form of a viscous oil, the ¹H NMR analysis of which shows the disappearance of the allyl proton signals and the appearance of propyl group signals at 0.5 and 1.5 ppm. The disappearance of Si—H groups is confirmed by ²⁹Si NMR.

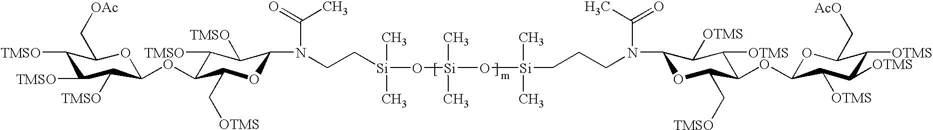

2/Hydrosilylation on Nonprotected Product A

0.5 g of N-acetyl allylcellobiose and 1.5 ml of isopropyl alcohol are introduced into a 50 ml three-necked reactor. The medium is heated to 70° C. in order to solubilize the cellobiose derivative and then 25 mg of ADAMS platinum catalyst are introduced. After the introduction of 0.745 g of 620H2 oil, the reaction medium is left at 70° C. for 4 hours and then treated as previously.

The NMR analysis confirms the expected structure with, however, the presence of DOR bonds characteristic of Si—O—C bonds.

Deprotection

The deprotection of the product C can be carried out at ambient temperature in a 50/50 methanol/THF medium in the presence of an acidic resin of the Amberlist type.

1-14. (canceled)

15. A grafted polymer having one of the following formulae: 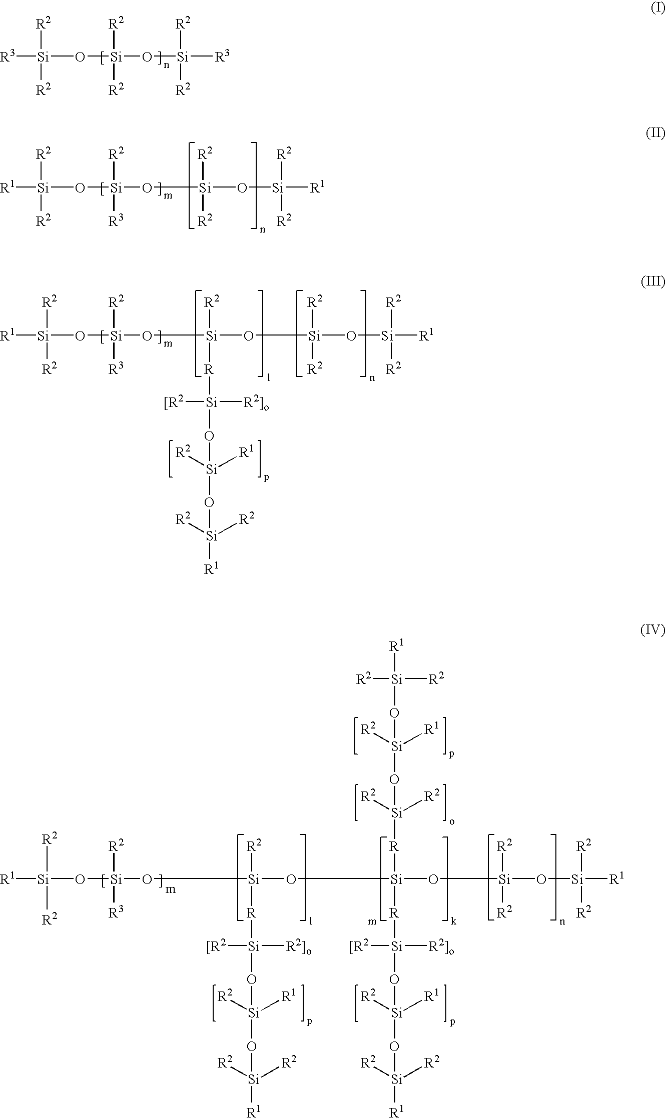
in which:
R², which is identical or different, is a hydrocarbon group, optionally a methyl group,
R³, which is identical or different, is a group of formula -L-G in which:
L is a divalent linker group having a nitrogen atom,
G is a glycoside having —OH groups,
R¹, which is identical or different, is an R² or R³ group,
R is a divalent group having an oxygen atom, optionally an —O— group,
n is an average number other than 0,
m is an average number greater than or equal to 0,
k and l are average numbers greater than or equal to 0, and
o and p, which are identical or different, are average numbers greater than or equal to 0.

16. The polymer as claimed in claim 15, wherein:
m+n is between 0 and 1000, optionally between 0 and 100, the ratio of m to n being between 1/1 and 1/100, optionally between 1/20 and 1/50, or
m+n+o+p is between 0 and 1000, optionally between 0 and 300, the ratio of n+o to m+p being between 1/1 and 1/100, optionally between 1/20 and 1/50.

17. The polymer as claimed in claim 15, wherein L is a divalent group having one of the following formulae:
NR⁴—(CH₂)_{q}—CH₂—CH₂—
NR⁴—(CH₂)_{q}—CH(CH₃)—
NR⁴—(CH₂)_{q}—CH═CH—, or
NR⁴—(CH₂)_{q}—C(CH₃)═CH—
in which
R⁴ is a hydrogen atom, an alkyl group or a protective group, and
q is an integer greater than or equal to 0.

18. The polymer as claimed in claim 15, wherein R³ is a group of formula G-NH—CH₂—CH₂—CH₂—, in which G has the formula G^{a}-G^{b}-, in which G^{a} is a glycoside, and G^{b} is a linker glycoside connected to the L group via an anomeric carbon.

19. The polymer as claimed in claim 15, wherein -G is a glycoside selected from the group consisting of:
glucose, fructose, sorbose, mannose, galactose, talose, allose, gulose, idose, glucosamine, mannoamine, galactosamine, glucuronic acid, rhamnose, arabinose, galacturonic acid, fucose, xylose, lyxose, ribose, palatinose,
maltose, gentiobiose, lactose, cellobiose, isomaltose, melibiose, laminaribiose, chitobiose, xylobiose, mannobiose, sophorose,
maltotriose, isomaltotriose, maltotetraose, maltopentaose, xyloglucan, maltoheptaose, mannotriose, manninotriose, maltodextrins, chitotriose,
starches having at least 5 dextrose equivalents, and galactomannans.

20. A method for preparing a grafted polymer as defined in claim 15, comprising the steps of:
a) preparing a graft compound of formula L′-G′, in which:
L′ is a group having a group that is ethylenically unsaturated, and having a nitrogen atom, and
G′ is a glycoside having optionally protected —OH groups,
b) grafting the graft compound by means of a method comprising a hydrosilylation step, said method comprising:
hydrosilylation of the graft compound with a backbone compound having one of the following formulae: 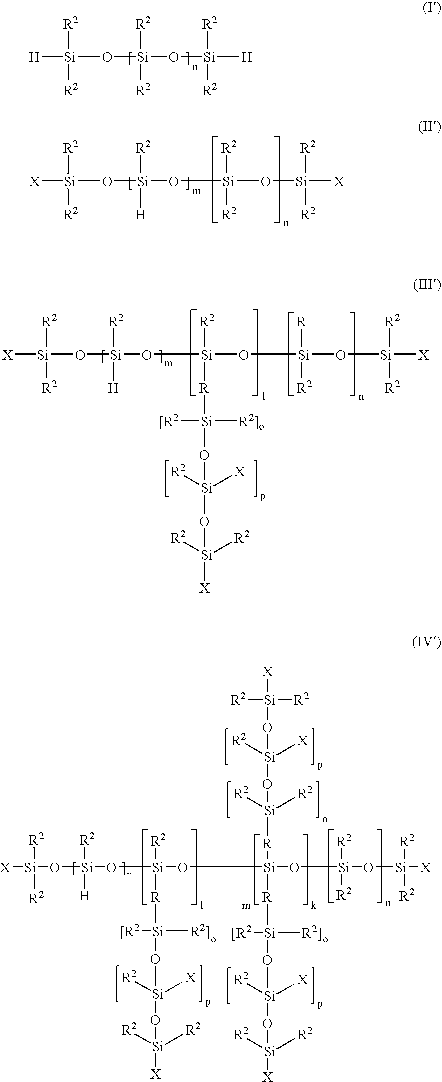
in which:
R², which is identical or different, is a hydrocarbon group, optionally a methyl group,
X, which is identical or different, is a hydrogen atom or an R²,
R is a divalent group having an oxygen atom, optionally an —O— group,
n is an average number other than 0,
m is an average number greater than or equal to 0,
k and l are average numbers greater than or equal to 0, and
o and p, which are identical or different, are average numbers greater than or equal to 0,
the reaction being carried out in a solvent which solubilizes or swells the graft and
the backbone, and
then, optionally, a redistribution, or
hydrosilylation of the graft compound with a backbone silane having the formula H—Si(OR⁵)₃₋ₐR⁵ₐ, in which R⁵, which is identical or different, is a hydrocarbon group, and a is equal to 1 or 2, the backbone silane optionally having the formula H—Si(—O—CH₂—CH₃)₂CH₃, the reaction being carried out in a solvent which solubilizes or swells the graft and the backbone, and then a redistribution in the presence of polyorganosiloxane, and
c) optionally, deprotecting —OH groups.

21. The method as claimed in claim 20, wherein the solvent is
an aprotic polar solvent, optionally DMF, DMAc,
a protic polar solvent, optionally methanol, IPA, TbuOH, or an apolar solvent, optionally toluene, hexane, xylene.

22. The method as claimed in claim 20, wherein the L′ group has one of the following formulae:
NR⁴—(CH₂)_{q′}—CH═CH₂, or
NR⁴—(CH₂)_{q′}—C≡CH₂
in which:
R⁴ is a hydrogen atom or an acetyl group —COCH₃, and
q′ is an integer greater than or equal to 0.

23. The method as claimed in claim 20, wherein the graft compound has the formula G′-N(COCH₃)—CH₂—CH═CH₂, in which G′ has the formula G^{a}-G^{b}- in which G^{a} is glycoside and G^{b} is a glycoside connected to the N atom by an anomeric carbon, said glycosides being optionally protected.

24. The method as claimed in claim 20, wherein the graft compound has the following formula:
G′-N(COCH₃)—CH₂—CH═CH₂
step a) comprising the following steps:
a1) reaction of the anomeric carbon of a glycoside G″ having free —OH groups with excess allylamine,
a2) elimination of the excess allylamine,
a3) reaction with acetic anhydride, so as to protect the nitrogen atom, and primary —OH groups of the glycoside, and
a4) optionally, protection of the other —OH groups of the glycoside.

25. The method as claimed in claim 24, wherein the free —OH groups are protected with an acetyl group —COCH₃ in step a), and in that the solvent in step b) is IPA.

26. The method as claimed in claim 20, wherein the G′ group is protected with trimethylsilyl groups.

27. The method as claimed in claim 20, wherein G′ is a glycoside selected from the group consisting of:
glucose, fructose, sorbose, mannose, galactose, talose, allose, gulose, idose, glucosamine, mannoamine, galactosamine, glucuronic acid, rhamnose, arabinose, galacturonic acid, fucose, xylose, lyxose, ribose, palatinose,
maltose, gentiobiose, lactose, cellobiose, isomaltose, melibiose, laminaribiose, chitobiose, xylobiose, mannobiose, sophorose,
maltotriose, isomaltotriose, maltotetraose, maltopentaose, xyloglucan, maltoheptaose, mannotriose, manninotriose, maltodextrins, chitotriose,
starches having at least 5 dextrose equivalents, galactomannans, and protected derivatives of these glycosides.

28. A compound of formula:
G″-N(COCH₃)—CH₂—CH═CH₂
in which G″ is a glycoside connected to the N atom via an anomeric carbon, the —OH groups of the glycosides being at least partially protected with acetyl groups of formula —COCH₃.
